(19) United States
(12) Patent Application Publication
(10) Pub. No.: US 2018/0024043 A1
(43) Pub. Date: Jan. 25, 2018

(54) METHOD FOR MONITORING MICROBIOLOGICALLY INDUCED CORROSION ON METAL SURFACES

(71) Applicants: Dow Global Technologies LLC, Midland, MI (US); Rohm and Haas Company, Collegeville, PA (US)

(72) Inventors: Selvanathan Arumugam, Blue Bell, PA (US); John W. Ashmore, Lansdale, PA (US); Tirthankar Ghosh, Oreland, PA (US); Heather R. Mcginley, Highland Park, IL (US)

(21) Appl. No.: 15/554,043

(22) PCT Filed: Feb. 26, 2016
(86) PCT No.: PCT/US2016/019671

§ 371 Date: Aug. 28, 2017

Related U.S. Application Data
(60) Provisional application No. 62/129,071, filed on Mar. 6, 2015.

(51) Int. Cl. G01N 17/00

(52) U.S. Cl. CPC G01N 17/006; C12N 1/00

(57) ABSTRACT

A method for monitoring microbiologically induced corrosion on metal surfaces in contact with a fluid. The method comprises adding to the fluid a silanol compound or silanol compound precursor having a group detectible by ultraviolet spectrophotometry or fluorescence spectroscopy, or which is isotopically enriched.

### BACKGROUND

This invention relates generally to a method for monitoring microbiologically induced corrosion on metal surfaces.

The use of silicates to inhibit corrosion of carbon steel surfaces is known. For example, U.S. Pat. No. 3,580,934 describes this use of silicate salts. However, the prior art does not disclose a method for monitoring microbiologically induced corrosion (MIC) on metal surfaces.

### STATEMENT OF INVENTION

The present invention is directed to a method for monitoring microbiologically induced corrosion on metal surfaces in contact with a fluid; said method comprising adding to the fluid a silanol compound or silanol compound precursor having a group detectible by ultraviolet spectrophotometry or fluorescence spectroscopy, or which is isotopically enriched.

The present invention is further directed to a method for monitoring microbiologically induced corrosion on metal surfaces in contact with a fluid; said method comprising adding to the fluid a silicate compound which is isotopically enriched.

### DETAILED DESCRIPTION

All percentages are weight percentages (wt %), and all temperatures are in ° C., unless otherwise indicated. All experimental work was performed at room temperature (“RT”), i.e., in the range from 20-25° C., unless specified otherwise. Concentrations in parts-per-million (“ppm”) are on a weight/weight basis.

As used herein, the term “metal surface” refers to the surface of a metal (preferably iron or steel), although this surface may have a natural coating of metal oxide.

As used herein, the term “silanol” refers to a compound having a hydroxyl group attached directly to a silicon atom. Preferably, the silanol has from one to three hydroxyl groups. Preferably, the group detectible by ultraviolet spectrophotometry or fluorescence spectroscopy is an organic group, i.e., one having carbon and hydrogen atoms and having no transition metal atoms, preferably one having only carbon, hydrogen, oxygen, sulfur and nitrogen atoms. In one preferred embodiment of the invention, the silanol is generated in situ in the fluid from a silanol compound precursor by adding a compound which hydrolyzes to a silanol (e.g., a trialkoxysilane or trichlorosilane). Preferably, the silanol compound precursor has formula XSi(G)₃ wherein G represents C₁-C₈ alkyl, C₁-C₈ alkoxy, chloro or a combination thereof provided that there is at least one alkoxy or chloro present; preferably X′CH₂Si(G)₃ preferably X′CH₂Si(OR)₃; wherein X or X′ represents a uv-active or fluorescent group and R is C₁-C₈ alkyl. Preferably, R is C₁-C₄ alkyl, preferably methyl. In one preferred embodiment, the silanol compound has the formula X′CH₂Si(R)₂OH, wherein R is C₁-C₈ alkyl; the silanol compound precursor for this compound has a chloro or alkoxy group in place of the hydroxyl group.

Preferably, the silanol or silanol compound precursor is added to the fluid and the level of free silanol compound in the fluid is monitored. Preferably, the level of silanol is monitored by detecting the uv-active or fluorescent group by known spectroscopic techniques.

Preferably, silanol or silanol compound precursor is added to the fluid to produce an initial silanol concentration of at least 10 ppm, preferably at least 30 ppm, preferably at least 50 ppm, preferably at least 70 ppm, preferably at least 90 ppm; preferably no more than 2 wt %, preferably no more than 1 wt %, preferably no more than 0.5 wt %, preferably no more than 0.1 wt %, preferably no more than 300 ppm, preferably no more than 250 ppm, preferably no more than 200 ppm. When the fluid is non-aqueous, e.g., oil or natural gas, preferably the initial silanol concentration is no more than 300 ppm, preferably no more than 250 ppm, preferably no more than 200 ppm.

Preferably, a uv-active group has a uv λₘₐₓ of at least 220 nm, preferably at least 230 nm, preferably at least 240 nm, preferably at least 250 nm; preferably no higher than 400 nm, preferably no higher than 370 nm, preferably no higher than 350 nm. Preferably, a fluorescent group has an excitation maximum from 220 to 650 nm and an emission maximum from 250 to 800 nm. A silanol or silicate which is isotopically enriched has at least one atom which is enriched in one isotope of that atom beyond the natural abundance level. The atom may be silicon, oxygen, hydrogen or another atom present in an organic substituent group of a silanol. An isotopically enriched organic substituent group preferably has the formula X′CH₂ wherein X′ is a group having from one to eighteen carbon atoms as well as hydrogen atoms, and which may also have oxygen, nitrogen and/or sulfur atoms. Preferably, X′ has no metal atoms. Preferably, X′ contains no atoms other than carbon, hydrogen, oxygen, nitrogen and sulfur. Silanols or silicates which are isotopically enriched may be detected by mass spectrometry.

Preferably, the concentration of silanol or silicate in the fluid at various times is determined by the appropriate method, depending on the nature of the group attached to the silanol or the isotopic enrichment of the silicate.

Silanols having uv-active groups include, e.g., bis(4-triethoxysilylpropoxy-3-methoxyphenyl)-1,6-heptadiene-3,5-dione; 3-(2,4-dinitrophenylamino)propyltriethoxysilane; 2-hydroxy-4-(3-methyldiethoxysilylpropoxy)diphenyl ketone; 2-hydroxy-4-(3-triethoxysilylpropoxy)diphenyl ketone; 7-triethoxysilylpropoxy-5-hydroxyflavone; 2-(2-triethoxysilylpropoxy-5-methylphenyl)benzotriazole; 3-(triethoxysilylpropyl)-p-nitrobenzamide and (R)—N-triethoxysilylpropyl-O-quinine-urethane. Silanols having fluorescent groups include, e.g., O-4-methylcoumarin-N-[3-(triethoxysilyl)propyl]carbamate and N-(triethoxysilylpropyl)dansylamide Preferably, uv-active or fluorescent groups are attached to the silanol silicon atom via a carbon chain, preferably —(CH₂)ₙ—, where n is from one to six, preferably from one to four.

The silanol is released as biofilm forms on the metal surface. The concentration of silanol in the fluid correlates with growth of biofilm and the rate of increase of the concentration correlates with the rate of growth. Growth of corrosive biofilm is known to correlate with the MIC process.

Preferably, the fluid has a water content of at least 0.01 wt %, preferably at least 0.1 wt %, preferably at least 0.5 wt %, preferably at least 1 wt %. Preferably, the fluid is crude oil, natural gas, water or an aqueous solution having at least 75% water (preferably at least 90%)

### EXAMPLES

Typical MIC Detection Procedure: Sterile anaerobic test vials were prepared. Serum bottles (10 mL bottle) were filled with culture media containing synthetic sea salts (35 g/L), glucose (1 g/L), sodium lactate (0.5 g/L), yeast extract (0.1 g/L), and sodium acetate (0.05 g/L). Peg-shaped carbon steel coupons were added to each one of the bottle. Serum bottles were sealed and autoclaved. For series 3 experiments, silica-treated coupons were used. Next, serum bottles were inoculated with SRB and APB at 1:100 dilutions from saturated cultures. For Series 1 experiments, soluble silicates (130-175 ppm) were introduced at this point. All samples were incubated at RT. Aliquots from the samples were drawn at different time points before complete formation of biofilm, centrifuged and prepared it for ICP analysis. The samples were left at RT to allow biofilm formation. When there was black color or precipitate in the coupon-containing inoculated samples, which is indicative of biofilm formation, more aliquots were drawn at different time points, centrifuged and prepared it for ICP analysis. The carbon steel surface was analyzed by XPS to determine the surface bound silica. For Series 2 experiments, soluble silicates (130-175 ppm) were introduced after the biofilm formation. The silica concentration in the supernatant is correlated the rate of biofilm action on metal bound silica. The amount of silica in supernatant after 24 hrs of introduction indicates the amount of silica that is immobilized on the coupon surface. The immobilized silica concentration is indicator of metal surface area available, in other words the extent of biofilm at the point of introduction.

Silica treatment of carbon steel coupons: A standard solution of soluble silicates of concentration 130-175 ppm was prepared. A freshly polished carbon steel coupon was immersed in the silicate solution and incubated at RT for 24 h. The metal coupon was removed from the silicate solution and washed with 5mL DI water thrice and the washings were combined with the incubate solution. The combined incubate solution was analyzed by ICP to determine the silica left over in the solution. From the silica remaining in the supernatant, the silica adsorbed to the surface was estimated.

Testing the influence other chemical and physical triggers on the silica release in MIC samples: The influence of infinite dilution, acid pH, basic pH, corrosion inhibitor such as alkybenzyl ammonium chloride, corroding chemicals such as hydrogen sulfide were monitored over time by following the procedure described below. Carbon steel coupons pre-treated with silica and the physically adsorbed silica was washed with DI water. The silica treated coupons were then placed in vial containing (1) DI water, (2) Instant Ocean (culture Media) (3) 0.1M HCl (pH 1), (4) 1 mM NaOH (pH 11), (5) corrosion inhibitor—alkybenzyl ammonium chloride (100 ppm in DI water), (6) hydrogen sulfide (150 μM) and (7) sterile water containing SRB bacteria. The supernatant and carbon steel coupon were respectively analyzed by ICP and XPS to determine released silica and the silica remained bound to coupon.

TABLE 1
Silica release profile Series 1-Silica introduced
along with SRB to untreated coupon

| | Incubation | Concentration |
|---|---|---|
| | Time | of Silica released |
| Sample | (days) | (ppm) |
| 1 | 1 | 2.99 |
| 2 | 4 | 12.84 |
| 3 | 7 | 42.66 |
| 4 | 21 | 94.23 |
| 5 | 28 | 118.1 |

TABLE 2
Silica release profile Series 2-Silica
introduced after biofilm formation

| | Incubation | Concentration of |
|---|---|---|
| | Time | Silica released |
| Sample | (days) | (ppm) |
| 1 | 7 | 52.71 |
| 2 | 8 | 69.42 |
| 3 | 10 | 83.78 |
| 4 | 21 | 107.2 |
| 5 | 28 | 126.5 |

TABLE 3
Silica release profile Series 3-Coupons were
pretreated with silica 24 h prior to SRB addition

| | Incubation | Concentration of |
|---|---|---|
| | Time | Silica released |
| Sample | (days) | (ppm) |
| 1 | 1 | 5 |
| 2 | 3 | 7.27 |
| 3 | 7 | 31.28 |
| 4 | 14 | 38.13 |
| 5 | 21 | 66.76 |
| 6 | 28 | 72.16 |

TABLE 4
Comparison of Silica release profile in presence
of various chemical and biological triggers

| | | Concentration of |
|---|---|---|
| | | Silica released |
| | | after 3 weeks |
| Sample | Trigger | incubation (%) |
| 1 | Dilution (DI water) | 3.7 |
| 2 | Instant Ocean | 5.6 |
| | (culture media) | |
| 3 | Acid (0.1M HCl) | 4.9 |
| 4 | Base (0.001M NaOH) | 6.1 |
| 5 | Alkybenzyl ammonium | 5.3 |
| | chloride (100 ppm) | |
| 6 | Hydrogen Sulfide (150 μM) | 7.1 |
| 7 | Sterile water containing | 89.6 |
| | SRM (biofilm) | |

TABLE 5
Comparison of Silica release profile in presence
of various chemical and biological triggers

| | | Δ Silica |
|---|---|---|
| | | Concentration |
| | | on carbon |
| | | steel |
| Sample | Trigger | coupon (%) |
| 1 | Dilution (DI water) | 3 |
| 2 | Instant Ocean | 0.0 |
| | (culture media) | |
| 3 | Acid (0.1M HCl) | 0.0 |
| 4 | Base (0.001M NaOH) | 11.2 |
| 5 | Alkybenzyl ammonium | 0.0 |
| | chloride (100 ppm) | |
| 6 | Hydrogen Sulfide (150 μM) | 2.9 |
| 7 | Sterile water containing | 100.0 |
| | SRM (biofilm) | |

The results demonstrate that the ferrosilicate/silica layer is stable at infinite dilution, acidic pH and basic pH. Exposure to chemicals such alkylbenzylammonium salts and hydrogen sulfide does not trigger the release of silicates from the carbon steel surface. However once biofilm starts growing on the surface, the chemically tethered silica is released back into the aqueous stream. The concentration of soluble silicates present in the aqueous stream serves as an indicator of available metal surface area and the rate at which silicates are released upon the action of corrosive biofilms serve as an indicator for occurrence of the MIC process at the surface.

1. A method for monitoring microbiologically induced corrosion on metal surfaces in contact with a fluid; said method comprising adding to the fluid a silanol compound or silanol compound precursor having a group detectible by ultraviolet spectrophotometry or fluorescence spectroscopy, or which is isotopically enriched.

2. The method of claim 1 in which said silanol or silanol compound precursor is added to the fluid to produce an initial silanol concentration from 10 ppm to 2 wt %.

3. The method of claim 2 in which the silanol compound precursor has formula XSi(G)₃, wherein G represents C₁-C₈ alkyl, C₁-C₈ alkoxy, chloro or a combination thereof provided that there is at least one alkoxy or chloro present; and wherein X represents a uv-active or fluorescent group.

4. The method of claim 3 in which a silanol compound precursor is added to the fluid.

5. The method of claim 4 in which the fluid has a water content from 0.01 to 100 wt %.

6. The method of claim 5 in which the silanol compound precursor has a group detectible by ultraviolet spectrophotometry or fluorescence spectroscopy

7. The method of claim 6 in which the fluid is crude oil having at least 0.05 wt % water.

8. The method of claim 7 in which the fluid is natural gas having at least 0.05 wt % water.

9. A method for monitoring microbiologically induced corrosion on metal surfaces in contact with a fluid; said method comprising adding to the fluid a silicate compound which is isotopically enriched.

10. The method of claim 9 further comprising monitoring a level of isotopically enriched silicate compound in the fluid.
